(19) United States
(12) Patent Application Publication
(10) Pub. No.: US 2024/0226582 A9
(43) Pub. Date: Jul. 11, 2024

(54) SUBCUTANEOUS DEVICE WITH ANCHORING ARMS

(71) Applicant: Calyan Technologies, Inc., Saint Paul, MN (US)

(72) Inventor: Yatheendhar D. Manicka, Woodbury, MN (US)

(21) Appl. No.: 18/493,567

(22) Filed: Oct. 24, 2023

Related U.S. Application Data
(60) Provisional application No. 63/418,894, filed on Oct. 24, 2022.

(51) Int. Cl. A61N 1/375; A61N 1/05; A61N 1/36; A61N 1/365

(52) U.S. Cl. CPC A61N 1/37518; A61N 1/0558; A61N 1/36135; A61N 1/365

(57) ABSTRACT

A subcutaneously implantable device includes a housing, a first anchoring arm attached to the housing that is configured to anchor the device to a muscle, a bone, and/or a first tissue, and a first prong with a proximal end attached to the housing and a distal end extending away from the housing that is configured to be positioned adjacent to or contact an organ, a nerve, or a second tissue. A first electrode is on the distal end of the first prong that is configured to contact the organ, the nerve, and/or the second tissue. Circuitry in the housing is in electrical communication with the first electrode and is configured to sense an electrical signal from the organ, the nerve, and/or the second tissue through the first electrode, and/or deliver electrical stimulation to the organ, the nerve, and/or the second tissue through the first electrode.

### CROSS-REFERENCE TO RELATED APPLICATION(S)

This application claims priority to U.S. Provisional Application No. 63/418,894, filed on Oct. 24, 2022, and entitled “Subcutaneous Device with Anchoring Arms,” the disclosure of which is incorporated by reference in its entirety.

### BACKGROUND

The present disclosure relates to implantable medical devices, and in particular, to a subcutaneous device.

Implantable medical devices include medical devices that are implanted in the body. Examples of implantable medical devices can include cardiac monitors, pacemakers, and implantable cardioverter-defibrillators, amongst many others. These implantable medical devices can receive signals from the body and use those signals for diagnostic purposes. These implantable medical devices can also transmit electrical stimulation or deliver drugs to the body for therapeutic purposes. For instance, a pacemaker can sense a heart rate of a patient, determine whether the heart is beating too fast or too slow, and transmit electrical stimulation to the heart to speed up or slow down different chambers of the heart. An implantable cardioverter-defibrillator can sense a heart rate of a patient, detect a dysrhythmia, and transmit an electrical shock to the patient.

Traditionally, cardiac monitors, pacemakers, and implantable cardioverter-defibrillators include a housing containing electrical circuitry. A proximal end of a lead is connected to the housing and a distal end of the lead is positioned in or on the heart. The distal end of the lead contains electrodes that can receive and transmit signals. Implantable medical devices such as cardiac monitors, pacemakers, and implantable cardioverter-defibrillators typically require invasive surgeries to implant the medical device in the body.

### SUMMARY

A subcutaneously implantable device includes a housing, a first anchoring arm attached to the housing that is configured to anchor the device to a muscle, a bone, and/or a first tissue, and a first prong with a proximal end attached to the housing and a distal end extending away from the housing that is configured to be positioned adjacent to or contact an organ, a nerve, or a second tissue. A first electrode is on the distal end of the first prong that is configured to contact the organ, the nerve, and/or the second tissue. Circuitry in the housing is in electrical communication with the first electrode and is configured to sense an electrical signal from the organ, the nerve, and/or the second tissue through the first electrode, and/or deliver electrical stimulation to the organ, the nerve, and/or the second tissue through the first electrode.

### BRIEF DESCRIPTION OF THE DRAWINGS

### Subcutaneous Device 2000

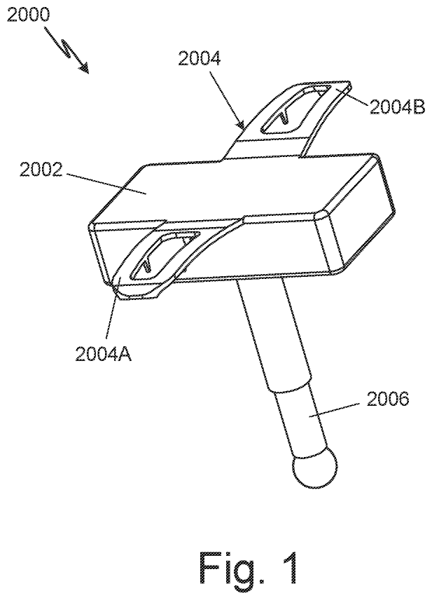

FIG. 1 is a perspective view of a first embodiment of a subcutaneous device.

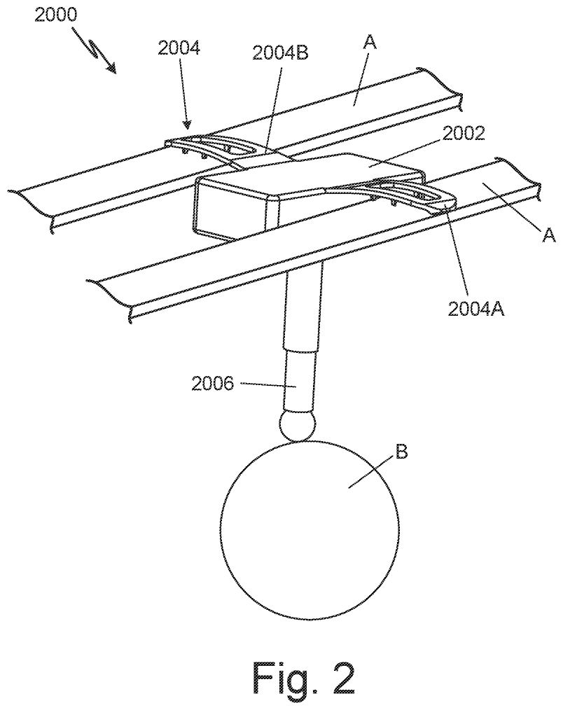

FIG. 2 is a perspective view of the first embodiment of the subcutaneous device anchored to a structural body component.

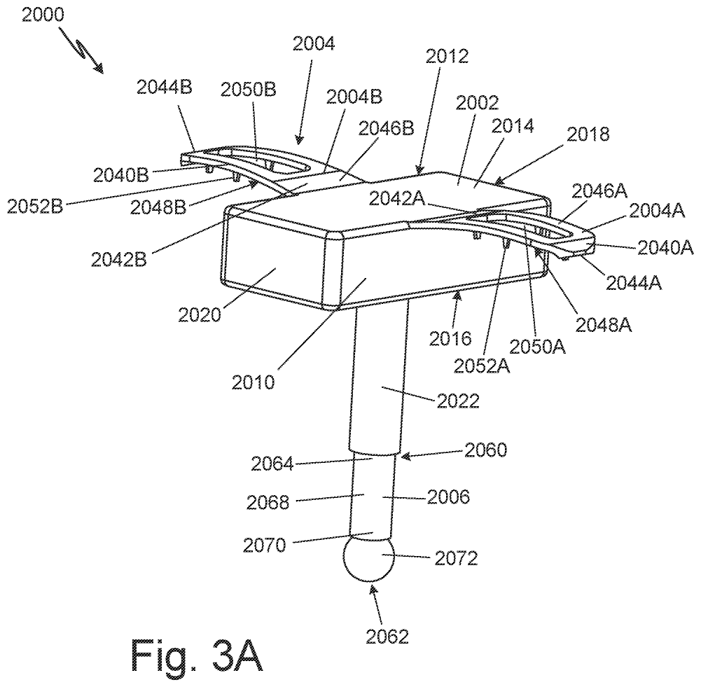

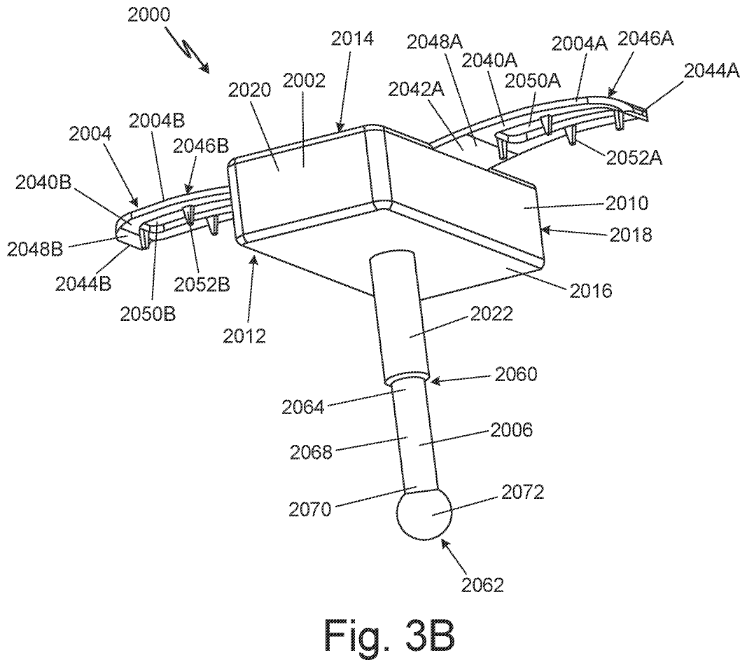

FIG. 3A is a top perspective view of the first embodiment of the subcutaneous device.

FIG. 3B is a bottom perspective view of the first embodiment of the subcutaneous device.

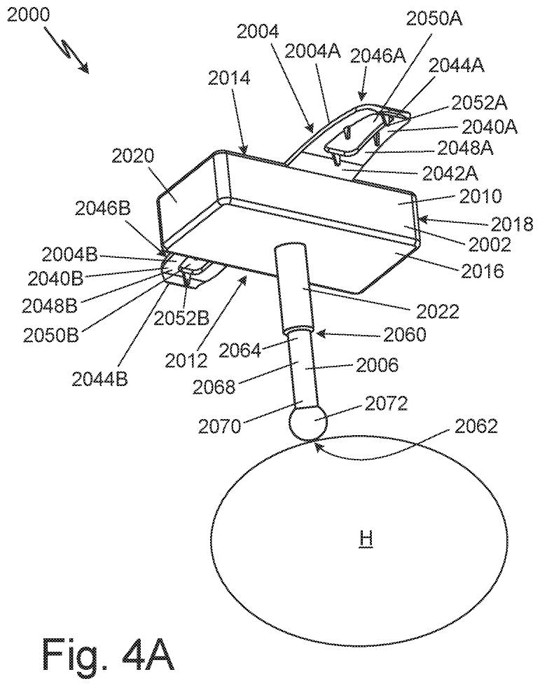

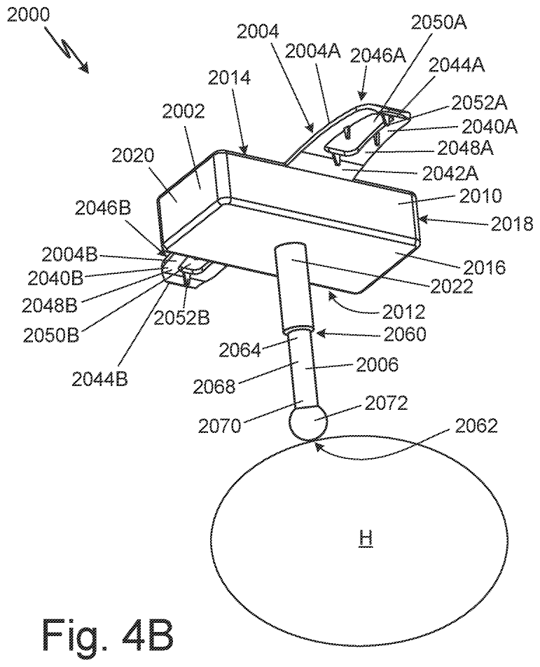

FIG. 4A is a bottom perspective view of the first embodiment of the subcutaneous device with the prong in an extended position and touching a heart.

FIG. 4B is a bottom perspective view of the first embodiment of the subcutaneous device with the prong in a retracted position and touching the heart.

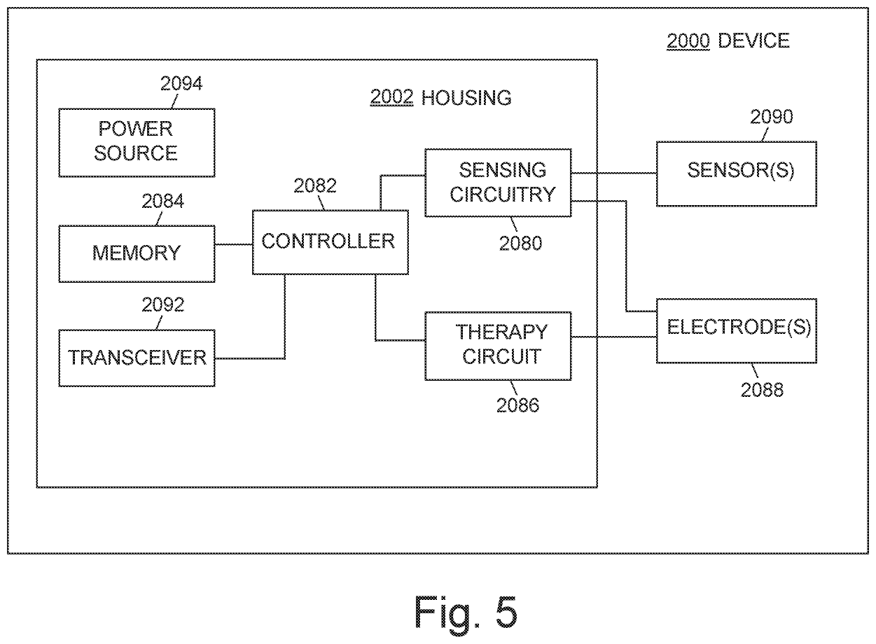

FIG. 5 is a functional block diagram of the first embodiment of the subcutaneous device.

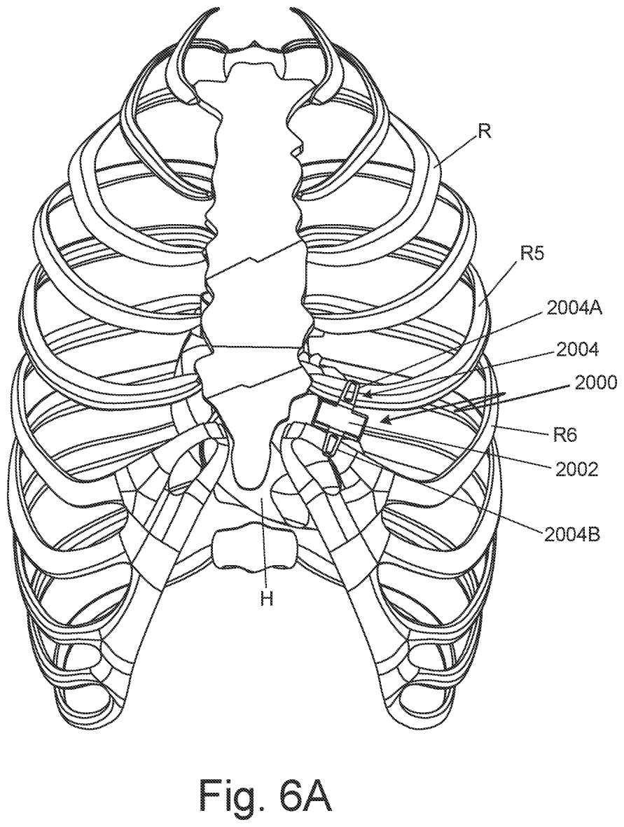

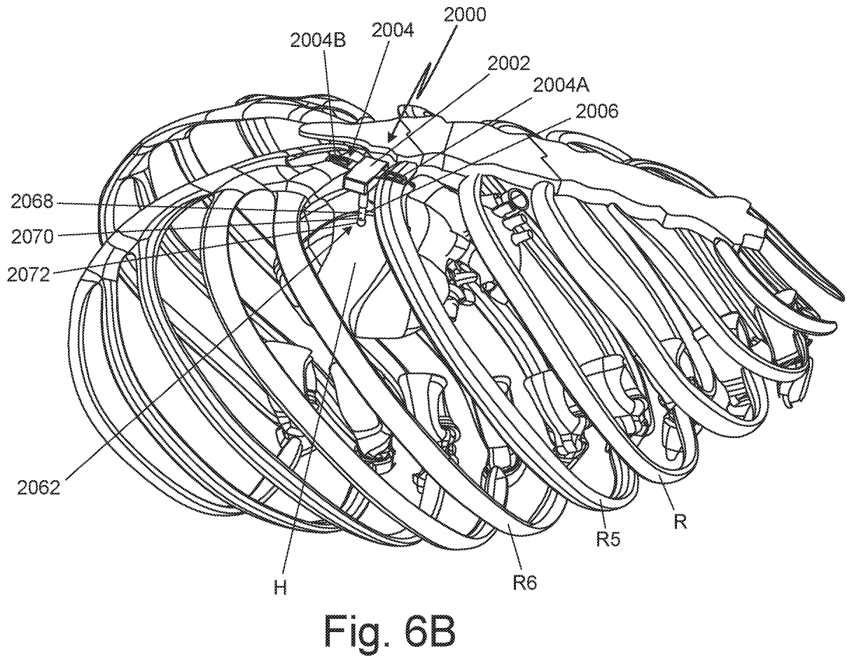

FIG. 6A is a front view of the first embodiment of the subcutaneous device positioned on ribs.

FIG. 6B is a side view of the first embodiment of the subcutaneous device positioned in the first location on the ribs and showing a positioning of the prong on a heart.

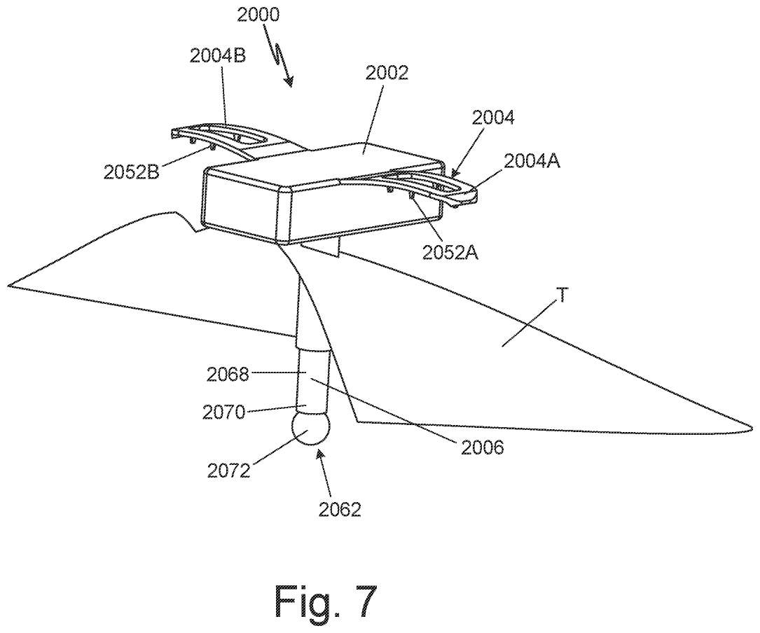

FIG. 7 is a perspective view of the first embodiment of the subcutaneous device with a prong extending through tissue.

### Subcutaneous Device 2100

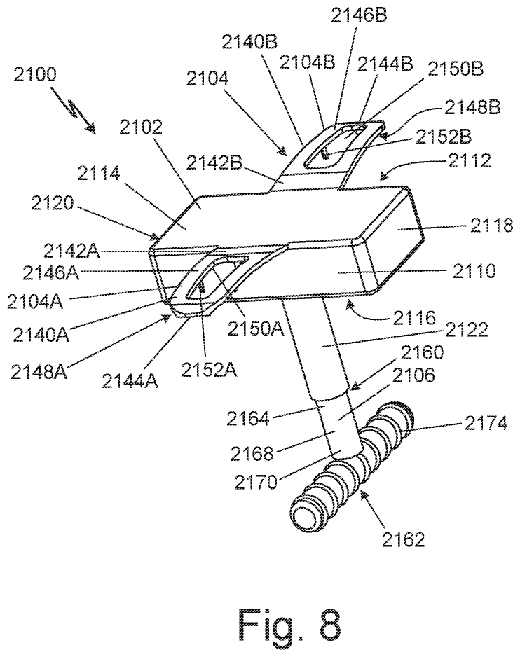

FIG. 8 is a perspective view of a second embodiment of a subcutaneous device.

### Subcutaneous Device 2200

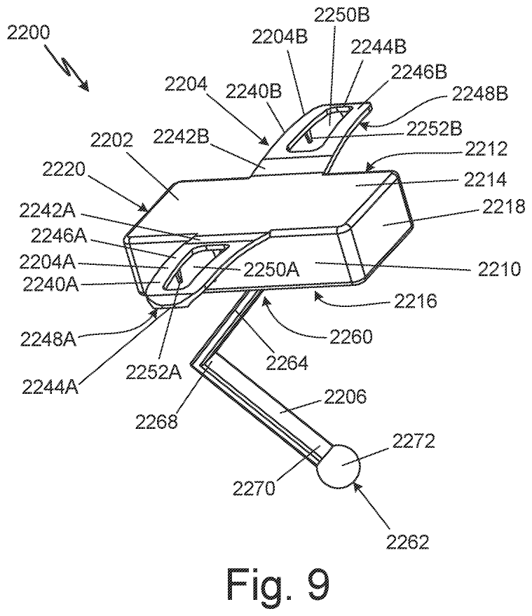

FIG. 9 is a perspective view of a third embodiment of a subcutaneous device.

### Subcutaneous Device 2300

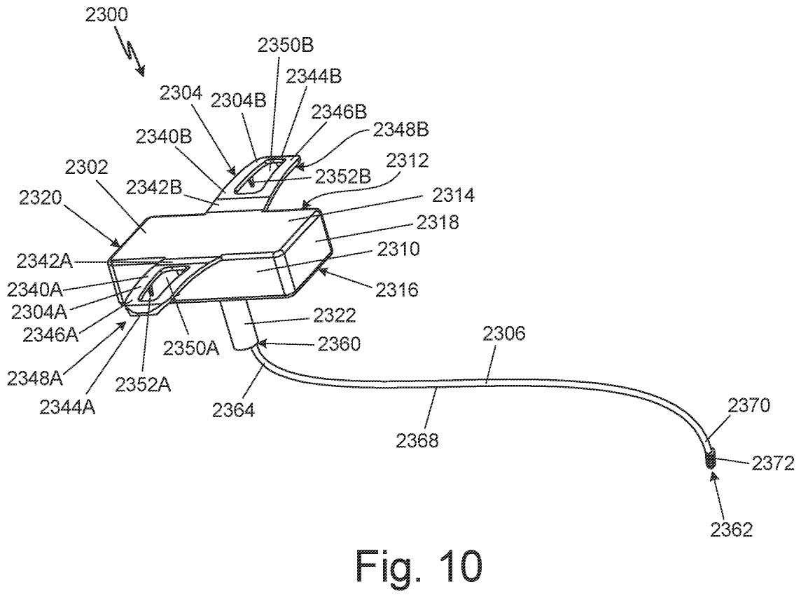

FIG. 10 is a perspective view of a fourth embodiment of a subcutaneous device.

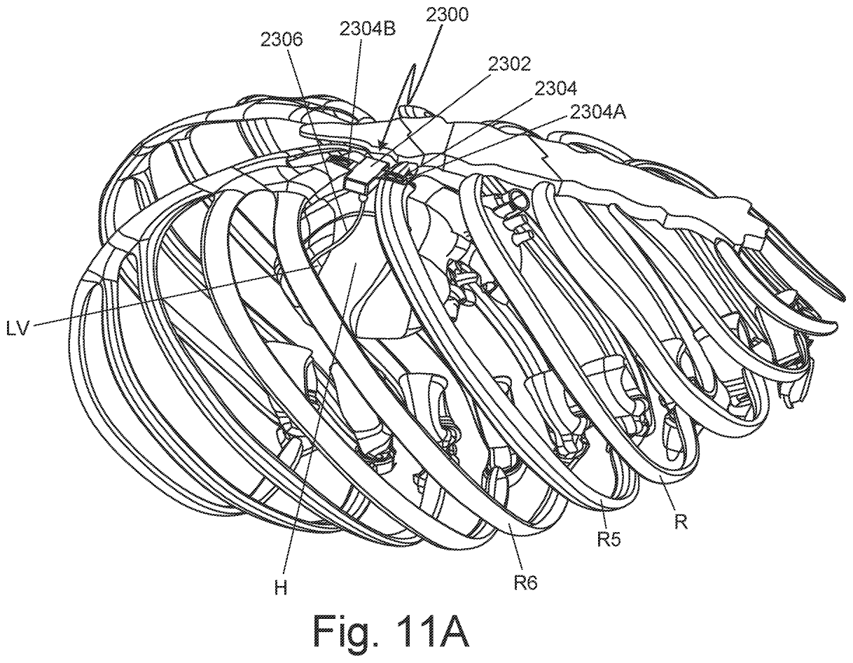

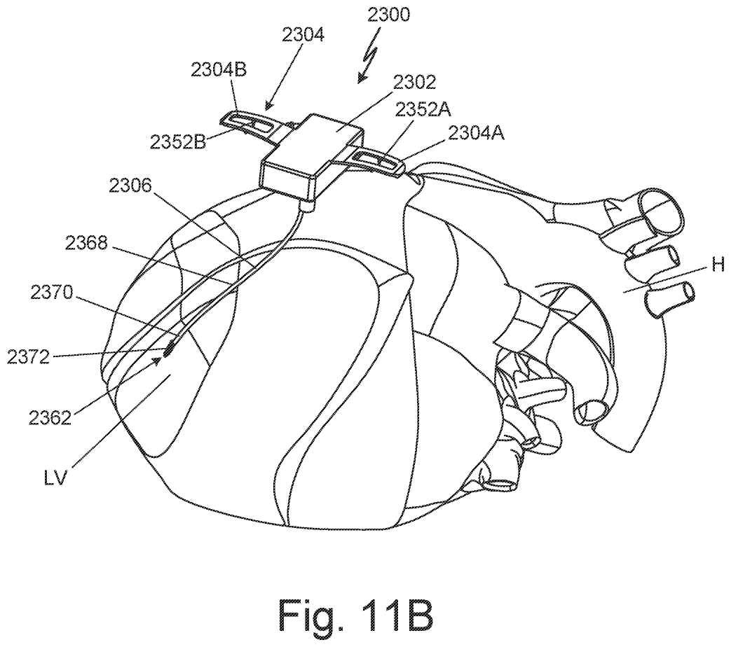

FIG. 11A is a side view of the fourth embodiment of the subcutaneous device positioned in the first location on the ribs and showing a positioning of the prong on a heart.

FIG. 11B is a side view of the fourth embodiment of the subcutaneous device and showing a positioning of the prong on the heart.

### Subcutaneous Device 2400

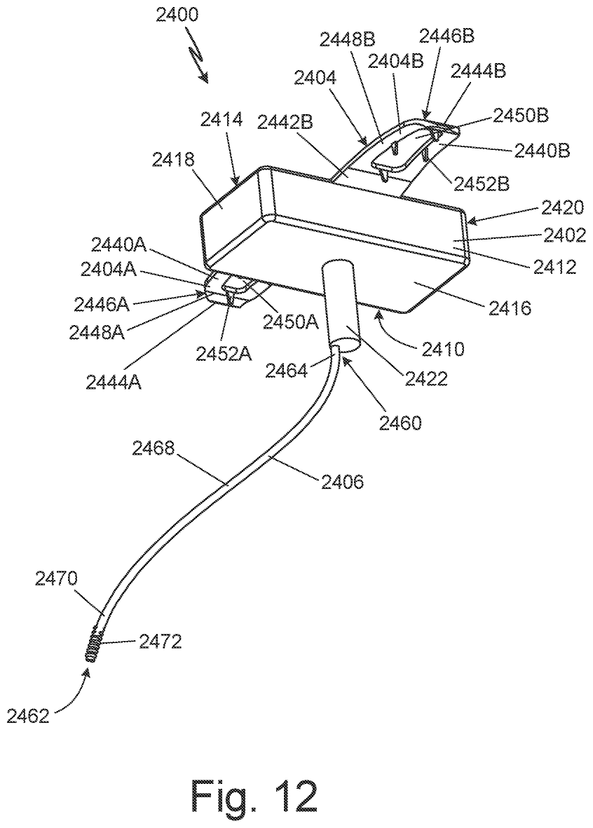

FIG. 12 is a perspective view of a fifth embodiment of a subcutaneous device.

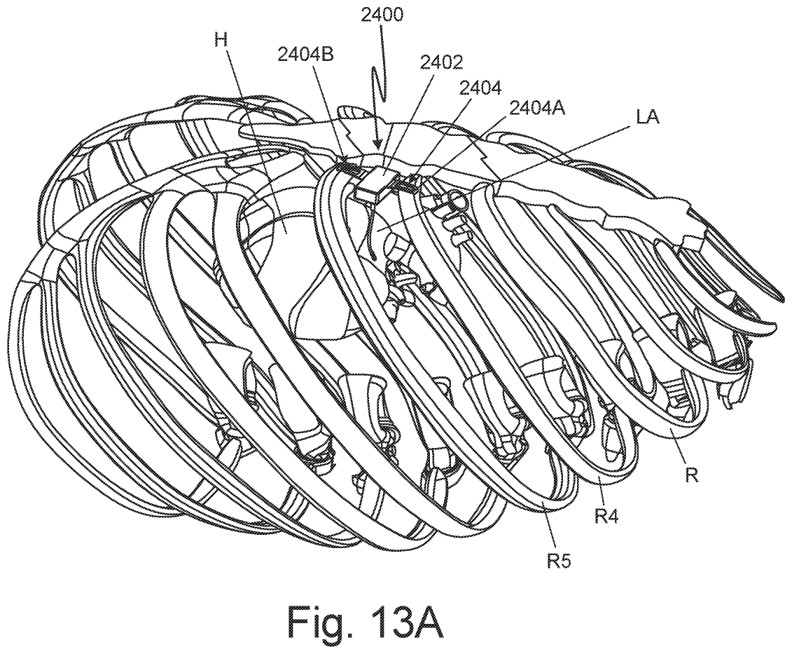

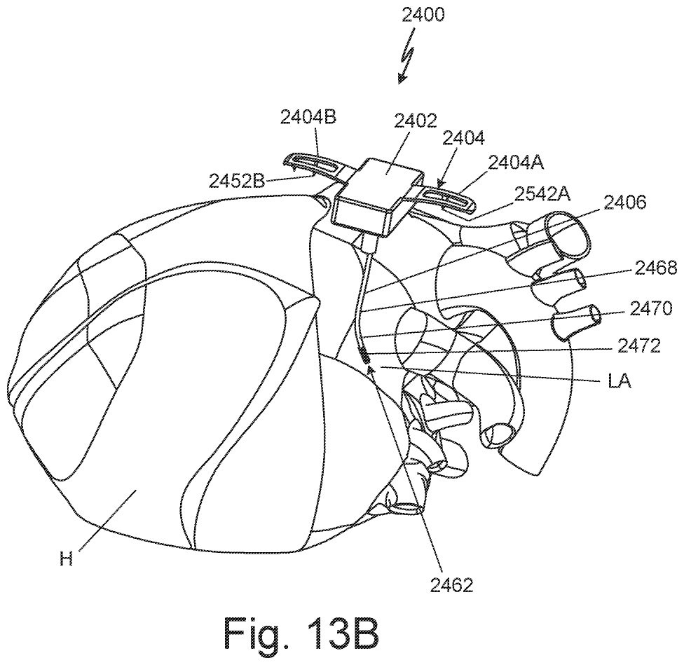

FIG. 13A is a side view of the fifth embodiment of the subcutaneous device positioned in a second location on the ribs and showing a positioning of the prong on a heart.

FIG. 13B is a side view of the fifth embodiment of the subcutaneous device and showing a positioning of the prong on the heart.

### Subcutaneous Devices 2500

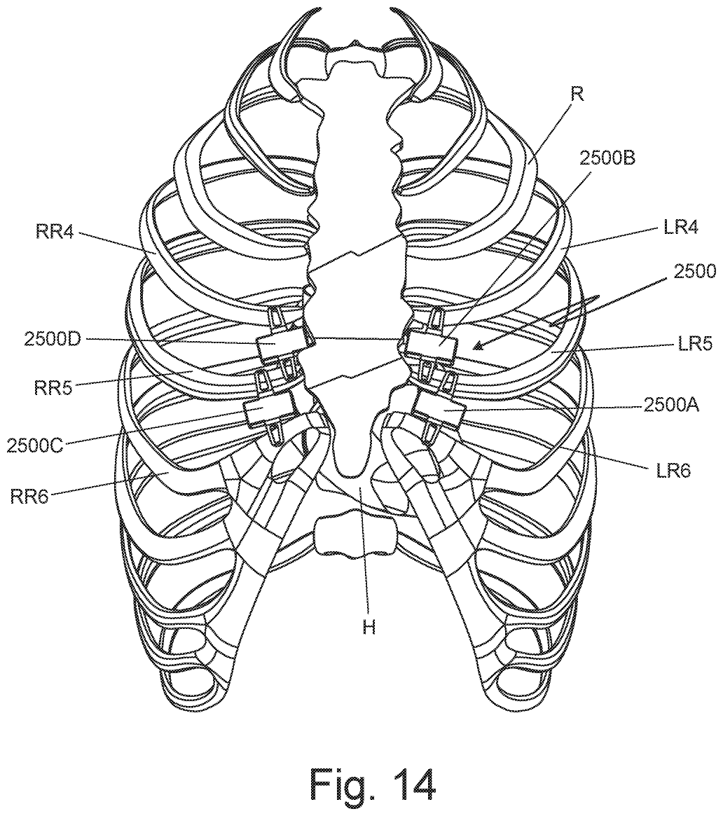

FIG. 14 is a front view of a plurality of subcutaneous devices positioned on ribs.

### DETAILED DESCRIPTION

In general, the present disclosure relates to a subcutaneous device that can be injected into a patient for monitoring, diagnostic, and therapeutic purposes. The subcutaneous device includes a housing that contains the electrical circuitry of the subcutaneous device, one or more anchoring arms attached to the housing, and one or more prongs extending away from the housing. The anchoring arms are configured to attach and anchor the subcutaneous device onto a muscle, a bone, or tissue. The prong extends away from the housing and a distal end of the prong comes into contact with an organ, a nerve, or tissue remote from the subcutaneous device.

The subcutaneous device can be a monitoring device, a diagnostic device, a pacemaker, an implantable cardioverter-defibrillator, a general organ/nerve/tissue stimulator, and/or a drug delivery device. A monitoring device can monitor physiological parameters of a patient. A diagnostic device can measure physiological parameters of a patient for diagnostic purposes. A pacemaker and an implantable cardioverter-defibrillator can sense a patient's heart rate and provide a therapeutic electrical stimulation to the patient's heart if an abnormality is detected. A pacemaker will provide an electrical stimulation to the heart in response to an arrhythmia, such as bradycardia, tachycardia, atrial flutter, and atrial fibrillation. The electrical stimulation provided by a pacemaker will contract the heart muscles to regulate the heart rate of the patient. An implantable cardioverter-defibrillator will provide an electrical stimulation to the heart in response to ventricular fibrillation and ventricular tachycardia, both of which can lead to sudden cardiac death. An implantable cardioverter-defibrillator will provide cardioversion or defibrillation to the patient's heart. Cardioversion includes providing an electrical stimulation to the heart at a specific moment that is in synchrony with the cardiac cycle to restore the patient's heart rate. Cardioversion can be used to restore the patient's heart rate when ventricular tachycardia is detected. If ventricular fibrillation is detected, defibrillation is needed. Defibrillation includes providing a large electrical stimulation to the heart at an appropriate moment in the cardiac cycle to restore the patient's heart rate. An implantable cardioverter-defibrillator can also provide pacing to multiple chambers of a patient's heart. A general organ/nerve/tissue stimulator can provide electrical stimulation to an organ, nerve, or tissue of a patient for therapeutic purposes. A drug delivery device can provide targeted or systemic therapeutic drugs to an organ, nerve, or tissue of a patient.

The subcutaneous device described in this disclosure can, in some embodiments, be anchored to a patient's ribs. The subcutaneous device can be anchored to the ribs so that the subcutaneous device is positioned in the intercostal space. When the subcutaneous device is anchored to the ribs, the one or more prongs of the subcutaneous device extend into the mediastinum and can contact an organ, a nerve, or a tissue, for example a heart.

### Subcutaneous Device 2000

FIG. 1 is a perspective view of subcutaneous device 2000. FIG. 2 is a perspective view of subcutaneous device 2000 anchored to structural body components A. Subcutaneous device 2000 includes housing 2002, anchoring arms 2004 (including first anchoring arm 2004A and second anchoring arm 2004B), and prong 2006. FIG. 2 shows structural body components A and remote body component B.

Subcutaneous device 2000 is a medical device that is anchored to structural body component(s) A. Structural body component(s) A may be a muscle, a bone, and/or a tissue of a patient. Subcutaneous device 2000 can be a monitoring device, a diagnostic device, a therapeutic device, or any combination thereof. For example, subcutaneous device 2000 can be a pacemaker device that is capable of monitoring a patient's heart rate, diagnosing an arrhythmia of the patient's heart, and providing therapeutic electrical stimulation to the patient's heart. Subcutaneous device 2000 includes housing 2002. Housing 2002 can contain a power source, a controller, a memory, a transceiver, sensors, sensing circuitry, therapeutic circuitry, and/or any other component of the medical device. Housing 2002 can also include one or more electrodes that are capable of sensing an electrical activity or physiological parameter of tissue surrounding housing 2002 and/or provide therapeutic electrical stimulation to the tissue surrounding housing 2002.

One or more anchoring arms 2004 are attached to housing 2002. Anchoring arms 2004 are anchoring mechanisms that are configured to anchor subcutaneous device 2000 to structural body component(s) A. Structural body component(s) A can be a muscle, a bone, and/or a tissue. Anchoring arms 2004 include first anchoring arm 2004A attached to a first side of housing 2002 and second anchoring arm 2004B attached to a second side of housing 2002 opposite of first anchoring arm 2004A. First anchoring arm 2004A and second anchoring arm 2004B can be anchored to the same structural body component A (i.e., the same muscle, bone, or tissue) or to different structural body components A (i.e., different muscles, bones, or tissues) positioned on opposite sides of housing 2002. Anchoring arms 2004 use an active fixation method such as sutures, tines, pins, screws, and/or any other suitable anchoring structure to secure anchoring arms 2004 to structural body component(s) A. In the embodiment shown in FIGS. 1-2, anchoring arms 2004 include tines that are configured to anchor subcutaneous device 2000 to structural body component(s) A.

Prong 2006 is connected to and extends away from housing 2002 of subcutaneous device 2000. Prong 2006 is configured to contact remote body component B that is positioned away from structural body component A. Remote body component B may be an organ, a nerve, or tissue of the patient. For example, remote body component B can include a heart, a lung, or any other suitable organ in the body. Prong 2006 includes one or more electrodes that are capable of sensing an electrical activity or physiological parameter of remote body component B and/or providing therapeutic electrical stimulation to remote body component B. Prong 2006 can be spring-loaded to allow it to extend and retract into housing 2002 when prong 2006 is in contact with remote body component B that moves, for example when remote body component B is a heart that beats.

In one example, subcutaneous device 2000 can be a pacemaker and the one or more electrodes on prong 2006 of subcutaneous device 2000 can sense the electrical activity of a heart. The sensed electrical activity can be transmitted to sensing circuitry and a controller in housing 2002 of subcutaneous device 2000. The controller can determine the heart rate of the patient and can detect whether an arrhythmia is present. If an arrhythmia is detected, the controller can send instructions to therapeutic circuitry to provide a therapeutic electrical stimulation to the heart. In this manner, subcutaneous device 2000 functions as a monitoring device, a diagnostic device, and a therapeutic device.

Subcutaneous device 2000 will be discussed in greater detail in relation to FIGS. 3A-7 below. Subcutaneous device 2000 will be discussed as a pacemaker that can be used for monitoring, diagnostics, and therapeutics in the discussion of FIGS. 3A-7 below. Subcutaneous device 2000 can also be used only for monitoring, diagnostics, or a combination of the two in alternate embodiments. Further, subcutaneous device 2000 can be a unipolar pacemaker or a bipolar pacemaker.

FIG. 3A is a top perspective view of subcutaneous device 2000. FIG. 3B is a bottom perspective view of subcutaneous device 2000. FIG. 4A is a bottom perspective view of subcutaneous device 2000 with prong 2006 in an extended position and touching heart H. FIG. 4B is a bottom perspective view of subcutaneous device 200 with prong 2006 in a retracted position and touching heart H. Subcutaneous device 2000 includes housing 2002, anchoring arms 2004 (including first anchoring arm 2004A and second anchoring arm 2004B), and prong 2006. Housing 2002 includes first side 2010, second side 2012, top side 2014, bottom side 2016, front end 2018, back end 2020, and receiving tube 2022. Anchoring arm 2004A includes body 2040A, housing side 2042A, outer side 2044A, top side 2046A, bottom side 2048A, opening 2050A, and tines 2052A. Anchoring arm 2004B includes body 2040B, housing side 2042B, outer side 2044B, top side 2046B, bottom side 2048B, opening 2050B, and tines 2052B. Prong 2006 includes proximal end 2060, distal end 2062, base portion 2064, arm portion 2068, therapeutic portion 2070, and electrode 2072. FIGS. 4A-4B further show heart H.

Subcutaneous device 2000 includes housing 2002, anchoring arms 2004, and prong 2006 as described in reference to FIGS. 1-2. Housing 2002 includes first side 2010, second side 2012, top side 2014, bottom side 2016, front end 2018, and back end 2020. First side 2010 is opposite of second side 2012; top side 2014 is opposite of bottom side 2016; and front end 2018 is opposite of back end 2020. Housing 2002 is substantially rectangular-shaped in the embodiment shown. In alternate embodiments, housing 2002 can be shaped as a cone, frustum, or cylinder. Housing 2002 can be made out of stainless steel, titanium, nitinol, epoxy, silicone, polyurethane with metallic reinforcements, or any other material that is suitable for non-porous implants. Housing 2002 can also include an exterior coating. In some embodiments, one or more electrodes can be positioned on housing 2002 to sense an electrical activity or physiological parameter of the tissue surrounding housing 2002 or provide therapeutic electrical stimulation to the tissue surrounding housing 2002.

Receiving tube 2022 of housing 2002 is connected to bottom side 2016 of housing 2002. Receiving tube 2022 is a cylindrically shaped body extending perpendicularly down from bottom side 2016 of housing 2002. Receiving tube 2022 is configured to receive prong 2006.

Anchoring arms 2004 include anchoring arm 2004A extending from first side 2010 of housing 2002 and anchoring arm 2004B extending from second side 2012 of housing 2002. Anchoring arms 2004 can be made out of stainless steel, titanium, nitinol, epoxy, silicone, polyurethane with metallic reinforcements, or any other material that is suitable for non-porous implants. Anchoring arm 2004A and anchoring arm 2004B are integrally formed with housing 2002 in the embodiment shown in FIGS. 3A-4B.

Anchoring arm 2004A includes body 2040A that is substantially rectangular shaped in the embodiment shown in FIGS. 3A-4B, but can have any suitable shape in alternate embodiments. Body 2040A extends from housing side 2042A to outer side 2044A. Housing side 2042A is opposite outer side 2044A. Anchoring arm 2004A is attached to housing 2002 at housing side 2042A of body 2040A. Outer side 2044A is an outer edge of body 2040A of anchoring arm 2004A. Body 2040A also has top side 2046A opposite bottom side 2048A. Opening 2050A extends through body 2040A from top side 2046A to bottom side 2048A. Opening 2050A is configured to allow anchoring arm 2004A to be sutured to a muscle, a bone, or a tissue in a patient to secure subcutaneous device 2000 to the muscle, the bone, or the tissue. Further, opening 2050A can receive additional fixation mechanisms, such as tines, pins, or screws, to secure subcutaneous device 2000 to the muscle, the bone, or the tissue. These additional fixation mechanisms can be made from bioabsorbable materials. In alternate embodiments, any number of openings 2050A may extend through body 2040A. In some embodiments, one or more electrodes can be positioned on anchoring arm 2004A to sense an electrical activity or physiological parameter of the tissue surrounding anchoring arm 2004A or provide therapeutic electrical stimulation to the tissue surrounding anchoring arm 2004A.

Anchoring arm 2004B includes body 2040B that is substantially rectangular shaped in the embodiment shown in FIGS. 3A-4B, but can have any suitable shape in alternate embodiments. Body 2040B extends from housing side 2042B to outer side 2044B. Housing side 2042B is opposite outer side 2044B. Anchoring arm 2004B is attached to housing 2002 at housing side 2042B of body 2040B. Outer side 2044B is an outer edge of body 2040B of anchoring arm 2004B. Body 2040B also has top side 2046B opposite bottom side 2048B. Opening 2050B extends through body 2040B from top side 2046B to bottom side 2048B. Opening 2050B is configured to allow anchoring arm 2004B to be sutured to a muscle, a bone, or a tissue in a patient to secure subcutaneous device 2000 to the muscle, the bone, or the tissue. Further, opening 2050B can receive additional fixation mechanisms, such as tines, pins, or screws, to secure subcutaneous device 2000 to the muscle, the bone, or the tissue. These additional fixation mechanisms can be made from bioabsorbable materials. In alternate embodiments, any number of openings 2050B may extend through body 2040B. In some embodiments, one or more electrodes can be positioned on anchoring arm 2004B to sense an electrical activity or physiological parameter of the tissue surrounding anchoring arm 2004B or provide therapeutic electrical stimulation to the tissue surrounding anchoring arm 2004B.

In the embodiment shown in FIGS. 3A-4B, tines 2052A extend from anchoring arm 2004A and tines 2052B extend from anchoring arm 2004B. Tines 2052A have first ends connected to bottom side 2048A of body 2040A and second ends that extend away from bottom side 2048A of body 2040A. Tines 2052B have first ends connected to bottom side 2048B of body 2040B and second ends that extend away from bottom side 2048B of body 2040B. In alternate embodiments, tines 2052A and tines 2052B may have first ends connected to any suitable portion of anchoring arm 2004A and anchoring arm 2004B, respectively. Tines 2052A and tines 2052B are thin and may be made of metal or any other suitable material. In the embodiment shown in FIGS. 3A-4B, anchoring arm 2004A has four tines 2052A, and anchoring arm 2004B has four tines 2052B. In alternate embodiments, anchoring arm 2004A and anchoring arm 2004B may have any number of tines 2052A and tines 2052B, respectively. Further, in alternate embodiments, any other suitable anchoring structures or active fixation methods may be used along with or instead of tines 2052A and tines 2052B. Tines 2052A and tines 2052B are configured to pierce and anchor to structural body component(s) A, such as a muscle, a bone, or a tissue, as seen in FIG. 2.

Anchoring arm 2004A and anchoring arm 2004B have a slight curve in the embodiment shown in FIGS. 3A-4B to fit to the muscle, the bone, or the muscle to which they are anchored. In alternate embodiments, anchoring arm 2004A and anchoring arm 2004B can be flat or have any degree of curvature to fit to any muscle, bone, or tissue.

When subcutaneous device 2000 is positioned on the muscle, the bone, or the tissue, anchoring arm 2004A and anchoring arm 2004B are pressed down onto the muscle, the bone, or the tissue. As anchoring arm 2004A and anchoring arm 2004B are pressed down onto the muscle, the bone, or the tissue, tines 2052A and tines 2052B attach to the muscle, the bone, or the tissue, to anchor anchoring arm 2004A and anchoring arm 2004B, respectively, to the muscle, the bone, or the tissue. Tines 2052A and tines 2052B will pierce the muscle, the bone, or the tissue in response to the pressure being placed on subcutaneous device 2000. Tines 2052A and tines 2052B can bend back around into the muscle, the bone, or the tissue when pressed onto the muscle, the bone, or the tissue, further securing and anchoring both anchoring arm 2004A and anchoring arm 2004B of subcutaneous device 2000 to the muscle, the bone, or the tissue. In the embodiment shown in FIGS. 3A-4B, prong 2006 connected to and extending away from housing 2002 can come into contact with heart H when housing 2002 is anchored to ribs with anchoring arm 2004A and anchoring arm 2004B.

Tines 2052A and tines 2052B are also removable from the muscle, the bone, or the tissue such that subcutaneous device 2000 is easily removable. The thin metal, or other suitable material, of tines 2052A and tines 2052B enables tines 2052A and tines 2052B to maintain flexibility. To remove anchoring arm 2004A and anchoring arm 2004B from structural body component(s) A, subcutaneous device 2000 is pulled away from structural body component(s) A. Pressure on anchoring arm 2004A and anchoring arm 2004B is reduced as subcutaneous device 2000 is pulled away from structural body component(s) A. Subcutaneous device 2000 can then be removed from the muscle, the bone, or the tissue and pulled out and removed from the body of the patient. Additional instruments, such as a scalpel or a cautery instrument may be used to assist in removal of subcutaneous device 2000 from the muscle, the bone, or the tissue.

Prong 2006 includes proximal end 2060 and distal end 2062 that is opposite of proximal end 2060. Proximal end 2060 of prong 2006 may have strain relief or additional material to support movement. Prong 2006 includes base portion 2064, arm portion 2068, and therapeutic portion 2070. A first end of base portion 2064 is aligned with proximal end 2060 of prong 2006, and a second end of base portion 2064 is connected to a first end of arm portion 2068. Base portion 2064 is a straight portion that is attached to housing 2002. Specifically, base portion 2064 is slidably received in receiving tube 2022 of housing 2002. Base portion 2064 extends into receiving tube 2022 of housing 2002 and is electrically connected to the internal components of housing 2002, for example with a feedthrough, to which prong 2006 is also connected. Base portion 2064 of prong 2006 is hermetically sealed in receiving tube 2022 of housing 2002.

The first end of arm portion 2068 is connected to the second end of base portion 2064, and a second end of arm portion 2068 is connected to a first end of therapeutic portion 2070. Arm portion 2068 is a straight portion in the embodiment shown in FIGS. 3A-4B that connects base portion 2064 to therapeutic portion 2070. Arm portion 2068 extends away from housing 2002 so that therapeutic portion 2070 is positioned outwards from housing 2002. In this embodiment, arm portion 2068 is a straight portion that extends away from housing 2002 in a first direction. In alternate embodiments, arm portion 2068 can be straight or curved and extend in any direction to reach any organ, nerve, or tissue, for example heart H as shown in FIGS. 4A-4B.

The first end of therapeutic portion 2070 is connected to the second end of arm portion 2068, and a second end of therapeutic portion 2070 is connected to electrode 2072 of prong 2006. Arm portion 2068 extends away from housing 2002 so that therapeutic portion 2070 is positioned away from housing 2002. Therapeutic portion 2070 can be positioned adjacent to or can come into contact with an organ, nerve, or tissue, for example heart H as shown in FIGS. 4A-4B.

Prong 2006 further includes electrode 2072. Electrode 2072 is shown as being connected to the second end of therapeutic portion 2070 and is aligned with distal end 2062 of prong 2006 in the embodiment shown in FIGS. 3A-4B. Electrode 2072 is a circular or rounded shaped electrode that can come into contact with an organ, nerve, or tissue, for example heart H, at any angle. In alternate embodiments, electrode 2072 can be positioned at any point on therapeutic portion 2070 and can have any shape and configuration. For example, electrode 2072 could be a coil electrode. The circular shape of electrode 2072 in FIGS. 3A-4B also allows electrode 2072 to safely come into contact with an organ, nerve, or tissue without piercing the organ, nerve, or tissue. For example, electrode 2072 is in contact with heart H in the embodiment shown in FIGS. 4A-4B and the rounded shape of electrode 2072 prevents electrode 2072 from piercing, puncturing, tearing, or otherwise damaging heart H. Prong 2006 is shown as having a single electrode 2072 in the embodiment shown in FIGS. 3A-4B. Prong 2006 can have any number of electrodes in alternate embodiments. Electrode 2072 is positioned on distal end 2062 of prong 2006 to sense an electrical activity or physiological status from an organ, nerve, or tissue, such as heart H, or provide therapeutic electrical stimulation to the organ, nerve, or tissue, such as heart H.

Prong 2006 is made of a stiff material so that it is capable of pushing through tissue in the body when subcutaneous device 2000 is implanted into a patient. Prong 2006 can be made out of a nickel titanium alloy, also known as Nitinol. Nitinol is a shape memory alloy with superelasticity, allowing prong 2006 to go back to its original shape and position if prong 2006 is deformed as subcutaneous device 2000 is implanted into a patient. Prong 2006 can also be made out of silicone, polyurethane, stainless steel, titanium, epoxy, polyurethane with metallic reinforcements, or any other material that is suitable for non-porous implants. As an example, prong 2006 can be made out of a composite made of polyurethane and silicone and reinforced with metal to provide spring stiffness.

Electrode 2072 of prong 2006 has a rounded shape to prevent prong 2006 from puncturing or damaging the organ, nerve, or tissue, such as heart H, it is contact with, along with other organs or tissue surrounding the organ, nerve, or tissue. For example, if electrode 2072 is in contact with heart H, electrode 2072 is rounded to prevent damage to heart H and also the pericardium, lungs, and other surrounding tissue. In alternate embodiments, electrode 2072 can have any shape that prevents it from puncturing or damaging the organ, nerve, or tissue, such as heart H, it is in contact with.

Prong 2006 is slidably received in receiving tube 2022 of housing 2002. The diameter of prong 2006 is sized to be slightly smaller than a diameter of a bore extending through receiving tube 2022 so that prong 2006 can be slidably received in receiving tube 2022. Prong 2006 can be spring loaded in receiving tube 2022 of housing 2002 to allow prong 2006 to extend out of and retract into receiving tube 2002 of housing 2002. Specifically, base portion 2064 of prong 2006 can extend out of and retract into receiving tube 2002 of housing 2002. As shown in FIG. 4A, prong 2006 is extended out of receiving tube 2002. This can be the normal state of prong 2006 when the spring is not compressed. As shown in FIG. 4B, prong 2006 is retracted into receiving tube 2022 and can compress a spring in housing 2002. Prong 2006 can be pushed into receiving tube 2022 as heart H beats. The spring in receiving tube 2022 can push prong 2006 outwards out of receiving tube 2022 and maintain pressure on prong 2006 to ensure prong 2006 maintains contact with heart H as heart H beats and contracts. Prong 2006 can slide into and out of receiving tube 2002 to allow prong 2006 to move as heart H beats. This allows prong 2006 to be flexible once it is positioned in the body, and prevents prong 2006 from piercing or tearing heart H and/or other organs or tissue surrounding heart H. The flexibility of prong 2006 permits electrode 2072 to maintain contact with an organ as the organ moves, for example with heart H as heart H beats.

Subcutaneous device 2000 is described here as having a single prong 2006. In alternate embodiments, subcutaneous device 2000 can include any number of prongs and those prongs can have any shape.

Anchoring arm 2004A includes tines 2052A and anchoring arm 2004B includes tines 2052B that attach to structural body component(s) A to sufficiently anchor subcutaneous device 2000 to structural body component(s) A, ensuring proper alignment of subcutaneous device 2000 with respect to structural body component(s) A and remote body component B. Tines 2052A and tines 2052B also allow for the removal of subcutaneous device 2000 from structural body component(s) A. Thus, subcutaneous device 2000 can be both securely implanted and easily removed for repair or replacement using less traumatic insertion and removal processes than a traditional implantable device.

Prong 2006 includes electrode 2072 on distal end 2062 that is configured to be in contact with an organ, nerve, or tissue, such as heart H. Subcutaneous device 2000 can function as a pacemaker. For example, prong 2006 can be shaped so that therapeutic portion 2070 of prong 2006 is positioned adjacent to or contacts the right ventricle, left ventricle, right atrium, or left atrium of heart H. Therapeutic circuitry in housing 2002 is electrically coupled to electrode 2072 on prong 2006. The therapeutic circuitry is configured to provide electrical stimulation to heart H. Subcutaneous device 2000 can function as a unipolar pacemaker, utilizing electrode 2072 on prong 2006 and an electrode on housing 2002 or on anchoring arms 2004. Further, subcutaneous device 2000 can function as a bipolar pacemaker, utilizing electrode 2072 on prong 2006 and a second electrode also positioned on prong 2006.

FIG. 5 is a functional block diagram of subcutaneous device 2000. Subcutaneous device 2000 includes housing 2002, sensing circuitry 2080, controller 2082, memory 2084, therapy circuitry 2086, electrode(s) 2088, sensor(s) 2090, transceiver 2092, and power source 2094.

Housing 2002 contains sensing circuitry 2080, controller 2082, memory 2084, and therapy circuitry 2086. Sensing circuitry 2080 receives electrical signals from the heart and communicates the electrical signals to controller 2082. Controller 2082 analyzes the electrical signals and executes instructions stored in memory 2084 to determine if there is an arrhythmia in the patient's heart rate. If controller 2082 determines that there is an arrhythmia, controller 2082 will send instructions to therapy circuitry 2086 to send electrical stimulation to the heart to regulate the heart rate of the patient. Sensing circuitry 2080 and therapy circuitry 2086 are both in communication with electrode(s) 2088. Electrode(s) 2088 can be positioned in housing 2002, anchoring arms 2004, and/or prong 2006 and are in contact with an organ, a nerve, or a tissue when subcutaneous device 2000 is implanted in a patient. Electrode(s) 2088 sense electrical signals from the organ, the nerve, or the tissue and provide electrical stimulation to the heart.

Controller 2082 is also in communication with sensor(s) 2090 through sensing circuitry 2080. Sensor(s) 2090 can be positioned in housing 2002 and/or prong 2006. Sensor(s) 2090 can be used with controller 2082 to determine physiological parameters of the patient. Controller 2082 is further in communication with transceiver 2092 that is positioned in housing 2002. Transceiver 2092 can receive information and instructions from outside of subcutaneous device 2000 and send information gathered in subcutaneous device 2000 outside of subcutaneous device 2000. Power source 2094 is also positioned in housing 2002 and provides power to the components in housing 2002, anchoring arms 2004, and prong 2006, as needed. Power source 2094 can be a battery that provides power to the components in housing 2002. In alternate examples, power source 2094 can be an external power source positioned outside of housing 2002.

Sensing circuitry 2080 is electrically coupled to electrode(s) 2088 via conductors extending through prong 2006 and into housing 2002. Sensing circuitry 2080 is configured to receive a sensing vector formed by electrode(s) 2088 and translate the sensing vector into an electrical signal that can be communicated to controller 2082. Sensing circuitry 2080 can be any suitable circuitry, including electrodes (including positive and negative ends), analog circuitry, analog to digital converters, amplifiers, microcontrollers, and power sources.

Controller 2082 is configured to implement functionality and/or process instructions for execution within subcutaneous device 2000. Controller 2082 can process instructions stored in memory 2084. Examples of controller 2082 can include any one or more of a microcontroller, a microprocessor, a digital signal processor (DSP), an application specific integrated circuit (ASIC), a field-programmable gate array (FPGA), or other equivalent discrete or integrated logic circuitry.

Memory 2084 can be configured to store information within subcutaneous device 2000 during operation. Memory 2084, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 2084 is a temporary memory, meaning that a primary purpose of memory 2084 is not long-term storage. Memory 2084, in some examples, is described as volatile memory, meaning that memory 2084 does not maintain stored contents when power to subcutaneous device 2000 is turned off. Examples of volatile memories can include random access memories (RAM), dynamic random access memories (DRAM), static random access memories (SRAM), and other forms of volatile memories. In some examples, memory 2084 is used to store program instructions for execution by controller 2082. Memory 2084, in one example, is used by software or applications running on subcutaneous device 2000 to temporarily store information during program execution.

Memory 2084, in some examples, also includes one or more computer-readable storage media. Memory 2084 can be configured to store larger amounts of information than volatile memory. Memory 2084 can further be configured for long-term storage of information. In some examples, memory 2084 can include non-volatile storage elements. Examples of such non-volatile storage elements can include magnetic hard discs, optical discs, floppy discs, flash memories, or forms of electrically programmable memories (EPROM) or electrically erasable and programmable (EEPROM) memories.

Controller 2082 can receive electrical signals from sensing circuitry 2080, analyze the electrical signals, and execute instructions stored in memory 2084 to determine whether an arrhythmia is present in the heart rate of a patient. If an arrhythmia is detected, controller 2082 can send instructions to therapy circuitry 2086 to deliver an electrical stimulation to the heart via electrode(s) 2088.

Therapy circuitry 2086 is electrically coupled to electrode(s) 2088 via conductors extending through prong 2006 and into housing 2002. Therapy circuitry 2086 is configured to deliver an electrical stimulation to the heart via electrode(s) 2088. Therapy circuitry 2086 will include a capacitor to generate the electrical stimulation. Therapy circuitry 2080 can be any suitable circuitry, including a microcontroller, power sources, capacitors, and digital to analog converters.

Controller 2082 can also receive information from sensor(s) 2090. Sensor(s) 2090 can include any suitable sensor, including, but not limited to, temperature sensors, accelerometers, pressure sensors, proximity sensors, infrared sensors, optical sensors, and ultrasonic sensors. The information from sensor(s) 2090 allows subcutaneous device 2000 to sense physiological parameters of a patient. For example, the data from the sensors can be used to calculate heart rate, heart rhythm, respiration rate, respiration waveform, activity, movement, posture, oxygen saturation, photoplethysmogram (PPG), blood pressure, core body temperature, pulmonary edema, and pulmonary wetness. The accelerometer can also be used for rate responsive pacing.

Subcutaneous device 2000 also includes transceiver 2092. Subcutaneous device 2000, in one example, utilizes transceiver 2092 to communicate with external devices via wireless communication. Subcutaneous device 2000, in a second example, utilizes transceiver 2092 to communication with other devices implanted in the patient via wireless communication. Transceiver 2092 can be a network interface card, such as an Ethernet card, an optical transceiver, a radio frequency transceiver, or any other type of device that can send and receive information. Other examples of such network interfaces can include Bluetooth, 3G, 4G, WiFi radio computing devices, Universal Serial Bus (USB), standard inductive coupling, low frequency medical frequency radio (MICS), ultra-wide band radio, standard audio, and ultrasonic radio. Examples of external devices that transceiver 2092 can communicate with include laptop computers, mobile phones (including smartphones), tablet computers, personal digital assistants (PDAs), desktop computers, servers, mainframes, cloud servers, or other devices. Other devices implanted in the body can include other implantable medical devices, such as other pacemakers, implantable cardioversion-defibrillators, nerve stimulators, and the like. Transceiver 2092 can also be connected to an antenna.

Subcutaneous device 2000 includes power source 2094 positioned in housing 2002. Subcutaneous device 2000 can also include a battery or device outside of housing 2002 that transmits power and data to subcutaneous device 2000 through wireless coupling or RF. Further, power source 2094 can be a rechargeable battery.

The internal components of subcutaneous device 2000 described above in reference to FIG. 5 are intended to be exemplary. Subcutaneous device 2000 can include more, less, or other suitable components. For example, when subcutaneous device 2000 is only used for diagnostics, subcutaneous device 2000 will not include therapy circuitry 2086. As a further example, subcutaneous device 2000 can function as a pacemaker without sensor(s) 2090.

FIG. 6A is a front view of subcutaneous device 2000 positioned on ribs R. FIG. 6B is a side view of subcutaneous device 2000 positioned in the first location on ribs R and showing a positioning of prong 2006 on heart H. FIG. 7 is a perspective view of subcutaneous device 2000 with prong 2006 extending through tissue T. Subcutaneous device 2000 includes housing 2002, anchoring arms 2004 (including anchoring arms 2004A and anchoring arms 2004B), and prong 2006. Anchoring arm 2004A includes tines 2052A. Anchoring arm 2004B includes tines 2052B. Prong 2006 includes distal end 2062, arm portion 2068, therapeutic portion 2070, and electrode 2072. FIGS. 6A-6B show ribs R, including fifth rib R5 and sixth rib R6, and heart H. FIG. 7 shows tissue T.

Subcutaneous device 2000 includes housing 2002, anchoring arms 2004, and prong 2006 as described above in reference to FIGS. 1-5. Subcutaneous device 2000 is a pacemaker device in the embodiment shown in FIGS. 6A-7. In alternate embodiments, subcutaneous device 2000 can be a monitoring device, a diagnostic device, a defibrillator, an organ/nerve/tissue stimulator, or any other suitable medical device. Subcutaneous device 2000 can be anchored to ribs R of a patient. When subcutaneous device 2000 is anchored to ribs R, housing 2002 of subcutaneous device 2000 will be positioned in an intercostal space of the patient.

Subcutaneous device 2000 can be implanted with a simple procedure where subcutaneous device 2000 is injected onto ribs R using a surgical instrument. An example surgical instrument and method of use thereof is shown in and described in U.S. application Ser. No. 17/020,356, filed on Sep. 14, 2020, and entitled “Clip Design for a Subcutaneous Device,” and having Attorney Docket No. C729-012018, the disclosure of which is incorporated by reference in its entirety. The surgical instrument can be designed to accommodate the shape of subcutaneous device 2000 and can be configured to push subcutaneous device 2000 out of the surgical instrument and onto ribs R. In the embodiment shown in FIGS. 6A-6B, subcutaneous device 2000 is anchored to fifth rib R5 and sixth rib R6 on the left side of the patient. Specifically, first anchoring arm 2004A is anchored to fifth rib R5 and second anchoring arm 2004B is anchored to sixth rib R6. In alternate embodiments, subcutaneous device 2000 can be anchored to the fourth rib and the fifth rib on the left side of the patient, the fifth rib and the sixth rib on the right side of the patient, the fourth rib and the fifth rib on the right side of the patient, or any other adjacent ribs. A small incision is made over ribs R of the patient. The surgical instrument that is pre-loaded with subcutaneous device 2000 will be inserted through the incision and advanced to ribs R. Anatomical markers can be used to guide subcutaneous device 2000 towards ribs R. For example, housing 2002 of subcutaneous device 2000 is directed toward the intercostal space between fifth rib R5 and sixth rib R6 to the patient's left side of ribs R, which directs prong 2006 to towards a left ventricle of heart H. As a result, subcutaneous device 2000 can be injected in a single direction, minimizing patient trauma. The surgical instrument pre-loaded with subcutaneous device 2000 will be positioned over ribs R, in this embodiment fifth rib R5 and sixth rib R6, and the surgical instrument can be activated to push anchoring arms 2004 of subcutaneous device 2000 on ribs R, in this embodiment fifth rib R5 and sixth rib R6. Cardiac catheterization labs are not needed to deploy subcutaneous device 2000. Housing 2002 will be positioned in the intercostal space between fifth rib R5 and sixth rib R6. When subcutaneous device 2000 is deployed onto ribs R, housing 2002 will be pushed into the intercostal space and prong 2006 of subcutaneous device 2000 will move through the mediastinum. Prong 2006 is made of a stiff material to allow it to push through the tissue in the mediastinum.

As shown in FIG. 7, prong 2006 can push through tissue T in a patient's body. The stiffness of prong 2006 allows it to dissect through tissue T and extend towards an organ, nerve, or tissue, such as heart H, targeted for sensing or treatment. Prong 2006 can be compressed into receiving tube 2022 of housing 2002 as it pushes through tissue T. The compressive strength of the spring in housing 2002 can be selected to push prong 2006 out from receiving tube 2022 of housing 2002 as it tunnels through tissue T. In some embodiments, a separate dissector can be used to create a tunnel in tissue T through which prong 2006 can extend.

Subcutaneous device 2000 can then be anchored to ribs R with anchoring arms 2004. Anchoring arms 2004 will hold subcutaneous device 2000 in position on ribs R. Tines 2052A and tines 2052B contact and connect to ribs R to anchor subcutaneous device 2000 to ribs R. Tines 2052A and tines 2052B dig into the rib tissue, muscle, and/or bone based on the amount of pressure placed on anchoring arms 2004. Under pressure, anchoring arms 2004 can be pushed onto ribs R such that tines 2052A and tines 2052B bend back around into ribs R. Anchoring arms 2004 anchor subcutaneous device 2000 to ribs R.

Anchoring arms 2004 hold subcutaneous device 2000 in position on ribs R. When subcutaneous device 2000 is anchored to ribs R, prong 2006 will extend from housing 2002 and contact heart H. Specifically, electrode 2072 of prong 2006 will come into contact with the pericardium. The pericardium is the fibrous sac that surrounds heart H. In the embodiment shown in FIGS. 6A-7, electrode 2072 will be positioned on the portion of the pericardium that surrounds a left ventricle of heart H. An electrical stimulation can be applied to the left ventricle of heart H, causing heart H to contract, by transmitting the electrical signal from electrode 2072 on distal end 2062 of prong 2006 through the pericardium and epicardium and into the myocardium of heart H. Prong 2006 can also sense electrical signals from heart H to determine a surface ECG of heart H. In alternate embodiments, prong 2006 can be shaped so that electrode 2072 of prong 2006 contacts pericardium over a right atrium, a right ventricle, or a left atrium of heart H.

As heart H beats, it will move in a vertical and a three-dimensional pattern. The spring-loaded retractability of prong 2006 into and out of receiving tube 2022 of housing 2002 provides flexibility to prong 2006 to allow prong 2006 to move with heart H as it beats. This will ensure that prong 2006 does not puncture or damage heart H.

Subcutaneous device 2000 can be implanted with a simple procedure where subcutaneous device 2000 is injected onto ribs R using a surgical instrument. The surgical procedure for implanting subcutaneous device 2000 is less invasive than the surgical procedure required for more traditional medical devices, as subcutaneous device 2000 is placed subcutaneously in the body. No leads need to be positioned in the vasculature of the patient, lowering the risk of thrombosis to the patient. Anchoring subcutaneous device 2000 to ribs R via anchoring arms 2004 ensures that subcutaneous device 2000 will not migrate in the patient's body. Maintaining the position of subcutaneous device 2000 in the body ensures that prong 2006 contacts heart H. Further, subcutaneous device 2000 is able to accurately and reliably determine a heart rate and other physiological parameters of the patient and provide therapeutic electrical stimulation to heart H, as subcutaneous device 2000 will not move in the patient's body. For instance, the ECG morphology will not change due to movement of subcutaneous device 2000 within the patient's body.

### Subcutaneous Device 2100

FIG. 8 is a perspective view of subcutaneous device 2100. Subcutaneous device 2100 includes housing 2102, anchoring arms 2104 (including first anchoring arm 2104A and second anchoring arm 2104B), and prong 2106. Housing 2102 includes first side 2110, second side 2112, top side 2114, bottom side 2116, front end 2118, back end 2120, and receiving tube 2122. Anchoring arm 2104A includes body 2140A, housing side 2142A, outer side 2144A, top side 2146A, bottom side 2148A, opening 2150A, and tines 2152A. Anchoring arm 2104B includes body 2140B, housing side 2142B, outer side 2144B, top side 2146B, bottom side 2148B, opening 2150B, and tines 2152B. Prong 2106 includes proximal end 2160, distal end 2162, base portion 2164, arm portion 2168, therapeutic portion 2170, and defibrillator coil 2174.

Subcutaneous device 2100 includes housing 2102, anchoring arms 2104, and prong 2106. Housing 2102 has the same general structure and design as housing 2002 of subcutaneous device 2000 shown in FIGS. 1-7. Anchoring arms 2104 have the same general structure and design as anchoring arms 2004 of subcutaneous device 2000 shown in FIGS. 1-7. The reference numerals that refer to the parts of housing 2102 and anchoring arms 2104 are incremented by one-hundred compared to the reference numerals that refer to the parts of housing 2002 and anchoring arms 2004 of subcutaneous device 2000 shown in FIGS. 1-7.

Prong 2106 generally includes the same parts as prong 2006 of subcutaneous device 2000 as shown in FIGS. 1-7, and the reference numerals that refer to the parts of prong 2106 are incremented by one-hundred compared to the reference numerals that refer to the parts of prong 2006 of subcutaneous device 2000 shown in FIGS. 1-7. However, prong 2106 has defibrillator coil 2174 instead of an electrode at distal end 2162. Arm portion 2168 extends away from bottom side 2116 of housing 2102. Therapeutic portion 2170 is a portion of prong 2106 adjacent to distal end 2162 of prong 2106 that is configured to be positioned near or come into contact with an organ, nerve, or tissue, for example a heart. Defibrillator coil 2174 is positioned on therapeutic portion 2170 adjacent to distal end 2162 of prong 2106.

Defibrillator coil 2174 is rod-shaped and extends outward from therapeutic portion 2170 of prong 2106. Defibrillator coil 2174 can sense signals from a heart and provide pacing or defibrillation to the heart. When an electrical signal is delivered to defibrillator coil 2174, defibrillator coil 2174 will create a vector between opposing ends of defibrillator coil 2174. A first end of defibrillator coil 2174 can serve as the negative electrode and a second end of defibrillator coil 2174 can serve as the positive electrode. This will create an electric field between opposite ends of defibrillator coil 2174 that will pass through a patient's heart to provide a high voltage electrical shock to the patient's heart.

Subcutaneous device 2100 can include a power source, a controller, a memory, a transceiver, sensors, sensing circuitry, therapeutic circuitry, electrodes, and/or any other component of a medical device. In the embodiment shown in FIG. 8, subcutaneous device 2100 is configured to be a defibrillator. Defibrillator coil 2174 can act as an electrode that senses the electrical activity of the heart, or one or more electrodes on housing 2102 or anchoring arms 2104 can sense an electrical activity of the heart. The sensed electrical activity can be transmitted to the sensing circuitry and the controller in housing 2102 of subcutaneous device 2100. The controller can determine the heart rate of the patient and can detect whether an abnormality is present. If an abnormality is detected, the controller can send instructions to therapeutic circuitry to provide a high voltage electrical shock to the heart using defibrillator coil 2174. In this manner, subcutaneous device 2100 functions as a monitoring device, a diagnostic device, and a therapeutic device. In alternate embodiments, subcutaneous device 2100 can function only as a monitoring device, a diagnostic device, or a therapeutic device, or any combinations thereof.

### Subcutaneous Device 2200

FIG. 9 is a perspective view of subcutaneous device 2200. Subcutaneous device 2200 includes housing 2202, anchoring arms 2204 (including first anchoring arm 2204A and second anchoring arm 2204B), and prong 2206. Housing 2202 includes first side 2210, second side 2212, top side 2214, bottom side 2216, front end 2218, and back end 2220. Anchoring arm 2204A includes body 2240A, housing side 2242A, outer side 2244A, top side 2246A, bottom side 2248A, opening 2250A, and tines 2252A. Anchoring arm 2204B includes body 2240B, housing side 2242B, outer side 2244B, top side 2246B, bottom side 2248B, opening 2250B, and tines 2252B. Prong 2206 includes proximal end 2260, distal end 2262, base portion 2264, arm portion 2268, therapeutic portion 2270, and electrode 2272.

Subcutaneous device 2200 includes housing 2202, anchoring arms 2204, and prong 2206. Housing 2202 has the same general structure and design as housing 2002 of subcutaneous device 2000 shown in FIGS. 1-7. However, housing 2200 does not include a receiving tube. Anchoring arms 2204 have the same general structure and design as anchoring arms 2004 of subcutaneous device 2000 shown in FIGS. 1-7. The reference numerals that refer to the parts of housing 2202 and anchoring arms 2204 are incremented by two-hundred compared to the reference numerals that refer to the parts of housing 2002 and anchoring arms 2004 of subcutaneous device 2000 shown in FIGS. 1-7.

Prong 2206 generally includes the same parts as prong 2006 of subcutaneous device 2000 as shown in FIGS. 1-7, and the reference numerals that refer to the parts of prong 2206 are incremented by two-hundred compared to the reference numerals that refer to the parts of prong 2006 of subcutaneous device 2000 shown in FIGS. 1-7. However, base portion 2264 of prong 2206 is attached to bottom side 2216 of housing 2202 instead of a receiving tube, and arm portion 2268 of prong 2206 has a different shape. Base portion 2264 of prong 2206 is attached to bottom side 2216 of housing 2202. In some embodiments, base portion 2264 can be received in a receiving structure on bottom side 2216 of housing 2202.

Arm portion 2268 extends away from bottom side 2216 of housing 2202. Arm portion 2268 has a generally v-shape. The v-shape of arm portion 2268 allows arm portion 2268 to act as a spring for prong 2206. In its natural state, a spring bias of arm portion 2268 forces distal end 2262 of prong 2206 away from bottom side 2216 of housing 2202. When electrode 2272 is in contact with a heart, the v-shape of arm portion 2268 of prong 2206 allows prong 2206 to move up and down as the heart beats, as arm portion 2268 functions as a spring that is pressing electrode 2272 down onto the heart. In some embodiments, prong 2206 may also be spring loaded in housing 2202 and extend from and retract into housing 2202.

Subcutaneous device 2200 can include a power source, a controller, a memory, a transceiver, sensors, sensing circuitry, therapeutic circuitry, electrodes, and/or any other component of a medical device. In the embodiment shown in FIG. 9, subcutaneous device 2200 is configured to be a single chamber pacemaker. Any one or combination of electrode 2272 or an electrode on housing 2202 or anchoring arms 2204 can sense the electrical activity of a heart. The sensed electrical activity can be transmitted to the sensing circuitry and the controller in housing 2202 of subcutaneous device 2200. The controller can determine the heart rate of the patient and can detect whether an arrhythmia is present. If an arrhythmia is detected, the controller can send instructions to therapeutic circuitry to provide a therapeutic electrical stimulation to the heart. Specifically, a therapeutic electrical stimulation can be provided to the left ventricle. In this manner, subcutaneous device 2200 functions as a monitoring device, a diagnostic device, and a therapeutic device. In alternate embodiments, subcutaneous device 2200 can function only as a monitoring device, a diagnostic device, a therapeutic device, or any combinations thereof.

### Subcutaneous Device 2300

FIG. 10 is a perspective view of subcutaneous device 2300. FIG. 11A is a side view of subcutaneous device 2300 positioned in the first location on ribs R and showing a positioning of prong 2306 on heart H. FIG. 11B is a side view of subcutaneous device 2300 and showing a positioning of prong 2306 on heart H. Subcutaneous device 2300 includes housing 2302, anchoring arms 2304 (including first anchoring arm 2304A and second anchoring arm 2304B), and prong 2306. Housing 2302 includes first side 2310, second side 2312, top side 2314, bottom side 2316, front end 2318, back end 2320, and receiving tube 2322. Anchoring arm 2304A includes body 2340A, housing side 2342A, outer side 2344A, top side 2346A, bottom side 2348A, opening 2350A, and tines 2352A. Anchoring arm 2304B includes body 2340B, housing side 2342B, outer side 2344B, top side 2346B, bottom side 2348B, opening 2350B, and tines 2352B. Prong 2306 includes proximal end 2360, distal end 2362, base portion 2364, arm portion 2368, therapeutic portion 2370, and electrode 2372. FIG. 11A shows ribs R, including fifth rib R5 and sixth rib R6. FIGS. 11A-11B shows heart H, including left ventricle LV.

Subcutaneous device 2300 includes housing 2302, anchoring arms 2304, and prong 2306. Housing 2302 has the same general structure and design as housing 2002 of subcutaneous device 2000 shown in FIGS. 1-7. Anchoring arms 2304 have the same general structure and design as anchoring arms 2004 of subcutaneous device 2000 shown in FIGS. 1-7. The reference numerals that refer to the parts of housing 2302 and anchoring arms 2304 are incremented by three-hundred compared to the reference numerals that refer to the parts of housing 2002 and anchoring arms 2004 of subcutaneous device 2000 shown in FIGS. 1-7.

Prong 2306 generally includes the same parts as prong 2006 of subcutaneous device 2000 as shown in FIGS. 1-7, and the reference numerals that refer to the parts of prong 2306 are incremented by three-hundred compared to the reference numerals that refer to the parts of prong 2006 of subcutaneous device 2000 shown in FIGS. 1-7. However, prong 2306 has a different shape. Arm portion 2368 extends away from receiving tube 2322 of housing 2302 and is shaped to extend towards left ventricle LV of heart H. Therapeutic portion 2370 is a portion of prong 2306 adjacent to distal end 2362 of prong 2306 that is configured to come into contact with left ventricle LV of heart H. Electrode 2372 positioned on therapeutic portion 2370 will also come into contact with left ventricle LV of heart H. Prong 2306 contacts left ventricle LV at a different location than prong 2006 of subcutaneous device 2000 shown in FIGS. 1-7.

Subcutaneous device 2300 is shown as being positioned in a first location between fifth rib R5 and sixth rib R6 in FIG. 11A. In the first location, prong 2306 is configured to extend towards and come into contact with left ventricle LV of heart H. In alternate embodiments, subcutaneous device 2300 can be anchored to the fourth rib and the fifth rib on the left side of the patient, the fifth rib and the sixth rib on the right side of the patient, the fourth rib and the fifth rib on the right side of the patient, or any other adjacent ribs.

Subcutaneous device 2300 can include a power source, a controller, a memory, a transceiver, sensors, sensing circuitry, therapeutic circuitry, electrodes, and/or any other component of a medical device. In the embodiment shown in FIGS. 10-11B, subcutaneous device 2300 is configured to be a single chamber pacemaker. Any one or combination of electrode 2372 or an electrode on housing 2302 or anchoring arms 2304 can sense the electrical activity of a heart. The sensed electrical activity can be transmitted to the sensing circuitry and the controller in housing 2302 of subcutaneous device 2300. The controller can determine the heart rate of the patient and can detect whether an arrhythmia is present. If an arrhythmia is detected, the controller can send instructions to therapeutic circuitry to provide a therapeutic electrical stimulation to the heart. Specifically, a therapeutic electrical stimulation can be provided to the left ventricle. In this manner, subcutaneous device 2300 functions as a monitoring device, a diagnostic device, and a therapeutic device. In alternate embodiments, subcutaneous device 2300 can function only as a monitoring device, a diagnostic device, a therapeutic device, or any combinations thereof.

### Subcutaneous Device 2400

FIG. 12 is a perspective view of subcutaneous device 2400. FIG. 13A is a side view of subcutaneous device 2400 positioned in the first location on ribs R and showing a positioning of prong 2406 on heart H. FIG. 13B is a side view of subcutaneous device 2400 and showing a positioning of prong 2406 on heart H. Subcutaneous device 2400 includes housing 2402, anchoring arms 2404 (including first anchoring arm 2404A and second anchoring arm 2404B), and prong 2406. Housing 2402 includes first side 2410, second side 2412, top side 2414, bottom side 2416, front end 2418, back end 2420, and receiving tube 2422. Anchoring arm 2404A includes body 2440A, housing side 2442A (not shown in FIGS. 12-13B), outer side 2444A, top side 2446A, bottom side 2448A, opening 2450A, and tines 2452A. Anchoring arm 2404B includes body 2440B, housing side 2442B, outer side 2444B, top side 2446B, bottom side 2448B, opening 2450B, and tines 2452B. Prong 2406 includes proximal end 2460, distal end 2462, base portion 2464, arm portion 2468, therapeutic portion 2470, and electrode 2472. FIG. 13A shows ribs R, including fourth rib R4 and fifth rib R5. FIGS. 13A-13B shows heart H, including right atrium RA.

Subcutaneous device 2400 includes housing 2402, anchoring arms 2404, and prong 2406. Housing 2402 has the same general structure and design as housing 2002 of subcutaneous device 2000 shown in FIGS. 1-7. Anchoring arms 2404 have the same general structure and design as anchoring arms 2004 of subcutaneous device 2000 shown in FIGS. 1-7. The reference numerals that refer to the parts of housing 2402 and anchoring arms 2404 are incremented by four-hundred compared to the reference numerals that refer to the parts of housing 2002 and anchoring arms 2004 of subcutaneous device 2000 shown in FIGS. 1-7.

Prong 2406 generally includes the same parts as prong 2006 of subcutaneous device 2000 as shown in FIGS. 1-7, and the reference numerals that refer to the parts of prong 2406 are incremented by four-hundred compared to the reference numerals that refer to the parts of prong 2006 of subcutaneous device 2000 shown in FIGS. 1-7. However, prong 2406 has a different shape. Arm portion 2468 extends away from receiving tube 2422 of housing 2402 and is shaped to extend towards left atrium LA of heart H. Therapeutic portion 2470 is a portion of prong 2406 adjacent to distal end 2462 of prong 2406 that is configured to come into contact with left atrium LA of heart H. Electrode 2472 positioned on therapeutic portion 2470 will also come into contact with left atrium LA of heart H.

Subcutaneous device 2400 is shown as being positioned in a second location between fourth rib R4 and fifth rib R5 in FIG. 13A. In the second location, prong 2406 is configured to extend towards and come into contact with left atrium LA of heart H. In alternate embodiments, subcutaneous device 2400 can be anchored to the fifth rib and the sixth rib on the left side of the patient, the fifth rib and the sixth rib on the right side of the patient, the fourth rib and the fifth rib on the right side of the patient, or any other adjacent ribs.

Subcutaneous device 2400 can include a power source, a controller, a memory, a transceiver, sensors, sensing circuitry, therapeutic circuitry, electrodes, and/or any other component of a medical device. In the embodiment shown in FIGS. 12-13B, subcutaneous device 2400 is configured to be a single chamber pacemaker. Any one or combination of electrode 2472 or an electrode on housing 2402 or anchoring arms 2404 can sense the electrical activity of a heart. The sensed electrical activity can be transmitted to the sensing circuitry and the controller in housing 2402 of subcutaneous device 2400. The controller can determine the heart rate of the patient and can detect whether an arrhythmia is present. If an arrhythmia is detected, the controller can send instructions to therapeutic circuitry to provide a therapeutic electrical stimulation to the heart. Specifically, a therapeutic electrical stimulation can be provided to the left ventricle. In this manner, subcutaneous device 2400 functions as a monitoring device, a diagnostic device, and a therapeutic device. In alternate embodiments, subcutaneous device 2400 can function only as a monitoring device, a diagnostic device, a therapeutic device, or any combinations thereof.

### Subcutaneous Devices 2500

FIG. 14 is a front view of plurality of subcutaneous devices 2500 positioned on ribs R. FIG. 14 shows plurality of subcutaneous devices 2500 (including subcutaneous device 2500A, subcutaneous device 2500B, subcutaneous device 2500C, and subcutaneous device 2500D), ribs R (including left fourth rib LR4, left fifth rib LR5, left sixth rib LR6, right fourth rib RR4, right fifth rib RR5, and right sixth rib RR6), and heart H.

FIG. 14 shows a plurality of subcutaneous devices 2500 positioned on ribs R. Subcutaneous devices 2500 have housings and anchoring arms having the same general structure and design as housing 2002 and anchoring arms 2004 of subcutaneous device 2000 shown in FIGS. 1-7. Subcutaneous devices 2500 also have prongs generally including the same parts as prong 2006 of subcutaneous device 2000 shown in FIGS. 1-7. The prongs of subcutaneous devices 2500 can have any suitable shape configured to contact different portions of heart H. For example, the prongs of subcutaneous devices 2500 can include prongs shaped like prong 2006 of subcutaneous device 2000 shown in FIGS. 1-7, prong 2206 of subcutaneous device 2200 shown in FIG. 9, prong 2306 of subcutaneous device 2300 shown in FIGS. 10-11B, and/or prong 2406 of subcutaneous device 2400 shown in FIGS. 12-13B. Additionally, prongs can have any other suitable shape and can be curved in any suitable configuration.

As shown in FIG. 14, subcutaneous device 2500A is anchored to left fifth rib LR5 and left sixth rib LR6. In one embodiment, subcutaneous device 2500A can have a prong that is configured to contact a left ventricle of heart H. Subcutaneous device 2500B is anchored to left fourth rib LR4 and left fifth rib LR5. In one embodiment, subcutaneous device 2500B can have a prong that is configured to contact a left atrium of heart H. Subcutaneous device 2500C is anchored to right fifth rib RR5 and right sixth rib RR6. In one embodiment, subcutaneous device 2500C can have a prong that is configured to contact a right ventricle of heart H. Subcutaneous device 2500D is anchored to right fourth rib RR4 and right fifth rib RR5. In one embodiment, subcutaneous device 2500D can have a prong that is configured to contact a right atrium of heart H.

Any one or more of subcutaneous devices 2500 can be used. For example, only one of subcutaneous device 2500A, subcutaneous device 2500B, subcutaneous device 2500C, and subcutaneous device 2500D can be used at a time. Alternatively, any combination of two, three, or four of subcutaneous device 2500A, subcutaneous device 2500B, subcutaneous device 2500C, and subcutaneous device 2500D can be used together. Further, subcutaneous device 2500A, subcutaneous device 2500B, subcutaneous device 2500C, and subcutaneous device 2500D can each have a prong having any shape and configured to contact any organ, nerve, or tissue.

Subcutaneous devices 2000, 2100, 2200, 2300, 2400, and 2500 disclose various pacemaker and defibrillator embodiments of the subcutaneous devices. Each of the pacemaker and defibrillator embodiments can also function as a monitoring and diagnostic device and/or a drug delivery device. Further, the features of each embodiment may be combined and/or substituted with features of any other embodiment, unless explicitly disclosed otherwise.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A subcutaneously implantable device comprising:
a housing;
a first anchoring arm attached to the housing that is configured to anchor the device to a muscle, a bone, and/or a first tissue;
a first prong with a proximal end attached to the housing and a distal end extending away from the housing that is configured to contact an organ, a nerve, or a second tissue;
a first electrode on the distal end of the first prong that is configured to contact the organ, the nerve, and/or the second tissue; and
circuitry in the housing in electrical communication with the first electrode that is configured to sense an electrical signal from the organ, the nerve, and/or the second tissue through the first electrode, and/or deliver electrical stimulation to the organ, the nerve, and/or the second tissue through the first electrode.

2. The device of claim 1, wherein the first anchoring arm comprises at least one tine configured to pierce the muscle, the bone, and/or the first tissue.

3. The device of claim 1, and further comprising:
a second anchoring arm attached to the housing that is configured to anchor the device to the muscle, the bone, and/or the first tissue.

4. The device of claim 3, wherein the first anchoring arm extends outward from a first side of the housing, and the second anchoring arm extends outward from a second side of the housing.

5. The device of claim 3, wherein the first anchoring arm and the second anchoring arm each comprises at least one tine configured to pierce the muscle, the bone, and/or the first tissue.

6. The device of claim 3, wherein the first anchoring arm and the second anchoring arm are integrally formed with the housing.

7. The device of claim 3, wherein the first anchoring arm and the second anchoring arm are configured to be anchored to a first rib and a second rib, respectively, in a patient.

8. The device of claim 1, wherein the first anchoring arm includes a body and an opening extending through the body.

9. The device of claim 8, wherein the opening in the body of the first anchoring arm can receive sutures, tines, pins, or screws to anchor the subcutaneous device to the muscle, the bone, and/or the first tissue.

10. The device of claim 1, wherein the first prong is made of a stiff material that is capable of pushing through tissue in a body of a patient.

11. The device of claim 1, wherein the first prong is slidably received in the housing.

12. The device of claim 1, wherein the first prong is slidably received in a receiving tube of the housing.

13. The device of claim 1, wherein the first prong is spring-loaded in the housing.

14. The device of claim 1, wherein the first prong is made out of a material selected from the group consisting of nickel titanium (Nitinol), silicone, polyurethane, stainless steel, titanium, epoxy, polyurethane with metallic reinforcements, and any combination thereof.

15. The device of claim 1, wherein a flexibility of the first prong is configured to permit the first electrode to maintain contact with an organ as the organ moves.

16. The device of claim 1, wherein the first prong further comprises:
a base portion slidably received in the housing;
an arm portion extending from the base portion and away from the housing; and
a therapeutic portion extending from the arm portion, wherein the electrode is connected to the therapeutic portion.

17. The device of claim 16, wherein the arm portion is a straight.

18. The device of claim 16, wherein the arm portion is curved.

19. The device of claim 1, wherein the first prong further comprises:
a base portion attached to the housing;
an arm portion extending from the base portion, wherein the arm portion has a v-shape and is configured to act as a spring for the first prong; and
a therapeutic portion extending from the arm portion, wherein the electrode is connected to the therapeutic portion.

20. The device of claim 1, wherein the electrode is configured to contact a right atrium, a right ventricle, a left atrium, or a left ventricle of a heart.
